(19) United States
(12) Patent Application Publication
(10) Pub. No.: US 2024/0068055 A1
(43) Pub. Date: Feb. 29, 2024

(54) QUALITATIVE, MULTIPLEX REVERSE TRANSCRIPTION PCR FOR THE DIFFERENTIAL DETECTION OF THE ORTHOPOX VIRUS MONKEYPOX

(71) Applicant: RenegadeXBio, PBC, BERKELEY, CA (US)

(72) Inventors: Craig Rouskey, Oakland, CA (US); Mikhail KULAK, San Francisco, CA (US)

(73) Assignee: RenegadeXBio, PBC, BERKELEY, CA (US)

(21) Appl. No.: 18/365,533

(22) Filed: Aug. 4, 2023

Related U.S. Application Data
(60) Provisional application No. 63/395,245, filed on Aug. 4, 2022.

(51) Int. Cl. C12Q 1/70

(52) U.S. Cl. CPC C12Q 1/701; C12Q 2600/16

(57) ABSTRACT

An assay and method for detecting a virus in a biological sample comprising a reverse transcription PCR assay that coverts viral messenger RNA to produce DNA; and amplifies the produced DNA in the presence of viral DNA. The assay is useful for detecting monkeypox virus.

### CROSS REFERENCE TO RELATED APPLICATIONS

This application claims the benefit of U.S. Provisional application No. 63/395,245 filed on Aug. 4, 2022, the contents of which are incorporated by reference herein.

### SEQUENCE LISTING

The instant application contains a Sequence Listing which has been submitted electronically in XML format and is hereby incorporated by reference in its entirety. Said XML copy, created on Nov. 2, 2023, is named P24952US01_SL.xml and is 25,281 bytes in size.

### TECHNOLOGICAL FIELD

This disclosure relates to a method for detecting an orthopoxvirus.

### BACKGROUND

Monkeypox Virus (MPXV) is a double-stranded DNA virus, a member of the Orthopoxvirus genus within the Poxviridae family. Poxviruses cause disease in humans and many other animals; infection typically results in the formation of lesions, skin nodules or disseminated rash. All orthopoxviruses (OPXV) are antigenically related and other species pathogenic to humans include cowpox virus and variola virus (causing smallpox, which has been eradicated). Vaccinia virus is also an OPXV that has been used as an attenuated vaccine and was a key tool for the eradication of smallpox, achieved in 1980.

Monkeypox virus transmits from subject to subject through close contact, aerosols, and through fomites. Primarily described in primates in 1958, it made its first appearance in the USA in 1959. Smallpox eradication programs mitigated disease spread. Given that the national smallpox vaccination program, a program which conferred resistance to monkeypox virus and smallpox, ceased in 1973, there is no viable herd immunity within the national community. Vaccinations received more than 10 years ago require boosters to reactivate immunity, and there are doses for 92M people in the USA.

MPXV is named due to its initial detection in monkeys and can primarily be found in rodents; however, the reservoir is undetermined. There are two known clades of MPXV, one endemic in Western Africa and one in the Congo Basin region.

After an incubation period ranging from 6 to 16 days, the typical presentation of monkeypox initiates with a short febrile prodromal period followed by progressive development of a classic rash with indurated and umbilicated (centrally depressed) lesions, starting on the head or face and progressing to the limbs and trunk.

Lesions progress all at the same stage from macules, to papules, to vesicles, to pustules and eventually to crusts which dry up and fall off after two to four weeks. There are often exanthems (sores or ulcers) in the mouth and lesions can affect the eyes and/or genital area. Because of the range of conditions that cause skin rashes and because clinical presentation may more often be atypical in this outbreak, it can be challenging to differentiate monkeypox solely based on the clinical presentation. Therefore, the decision to test should be based on both clinical and epidemiological factors, linked to an assessment of the likelihood of infection. Any individual meeting the definition for a suspected case of Monkeypox should be offered testing.

Unlike many other infectious diseases, there are treatments available for the illness. TPOXX® is an antiviral known to reverse lesion progression and offer relief from the excruciating pain caused by pustules that release virus into the periphery. By administering the drug early in infection, we can prevent transmission of the virus within exposed communities.

Given the current multiple detection of MXPV world-wide and looming outbreak of Monkeypox virus in the USA in 2022, and current limited availability of diagnostic tests that detect virus prior to lesion formation, there is a clear market need for testing-as-intervention assays. It has become increasingly clear that we need a high-throughput, ready to use diagnostic at scale that allows for early detection and intervention in viral diseases such as monkeypox virus.

### SUMMARY

To this end, we worked to optimize a multiplex real-time, reverse transcription PCR assay including not only RNA input, but DNA input as well in a single assay. The Reverse Transcription (RT) reaction converts messenger RNA molecules created during viral replication to DNA, which is then amplified in conjunction with assembled viral DNA allowing a 10× improvement in sensitivity, enabling earlier detection of monkeypox viruses in multiple sample types (blood, plasma, serum, saliva, oropharyngeal swabs, urine, and lesion swabs). By employing this technology, we can support earlier intervention in Monkeypox disease, and support test-to-treat methods that prevent disease spread by preventing pustule formation.

In one aspect, provided is an assay for detecting a virus in a biological sample, wherein the assay comprises a reverse transcription PCR assay that coverts viral messenger RNA to produce DNA; and amplifies the produced DNA in the presence of viral DNA.

In embodiments, the virus is an orthopoxvirus, notably the causative agent of Monkeypox.

Additional embodiments include the following.

The assay wherein a generic orthopox virus primer set and additional primers for clade-specific regions are included in the assay.

The assay wherein the additional primers allow for the determination of West African monkeypox virus or Congo Basin monkeypox virus.

The assay wherein two targets are virus-specific targeting two monkeypox conservative areas of the genome probes and an indigenous control targeting human RNase P gene area.

The assay wherein the virus-specific targeting probes are allocated within DNA-binding phosphoprotein (2) (I3L) and Protein F11 (F11L) genes of the monkeypox virus.

Another aspect provides a method for detecting a virus in a biological sample, wherein the method comprises converting viral messenger RNA to produce DNA in a reverse transcription PCR assay; and amplifying the produced DNA in the presence of viral DNA.

In embodiments, the virus is an orthopoxvirus, notably the causative agent of Monkeypox.

Additional embodiments of the method include the following.

The method wherein a generic orthopox virus primer set and additional primers for clade-specific regions are included in the assay.

The method wherein the additional primers allow for the determination of West African monkeypox virus or Congo Basin monkeypox virus.

The method wherein two targets are virus-specific targeting two monkeypox conservative areas of the genome probes and an indigenous control targeting human RNase P gene area.

The method wherein the virus-specific targeting probes are allocated within DNA-binding phosphoprotein (2) (I3L) and Protein F11 (F11L) genes of the monkeypox virus.

### BRIEF DESCRIPTION OF DRAWINGS

FIG. 1 shows a flow scheme of using the monkeypox assay according to an exemplary embodiment of the disclosed subject matter.

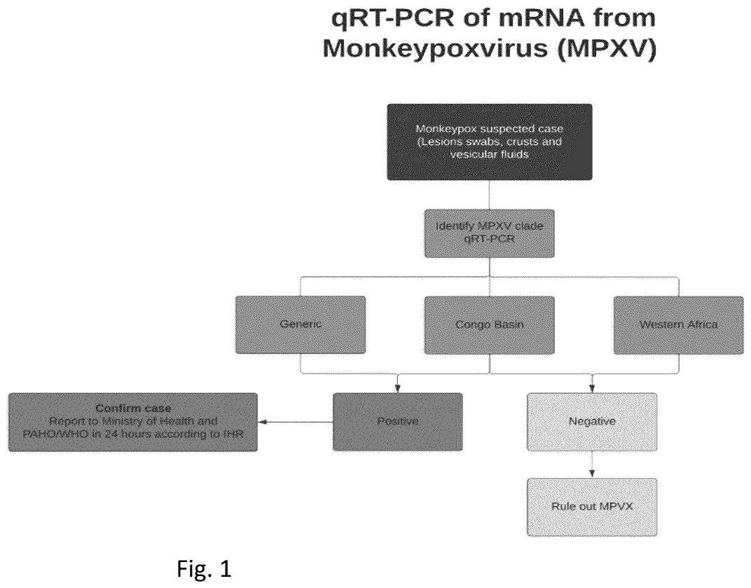

FIG. 2 shows the average difference in Ct-values based on method, qPCR versus RT-PCR according to an exemplary embodiment of the disclosed subject matter.

FIGS. 3A-B show aspects of the detection of monkeypox by the assay according to an exemplary embodiment of the disclosed subject matter.

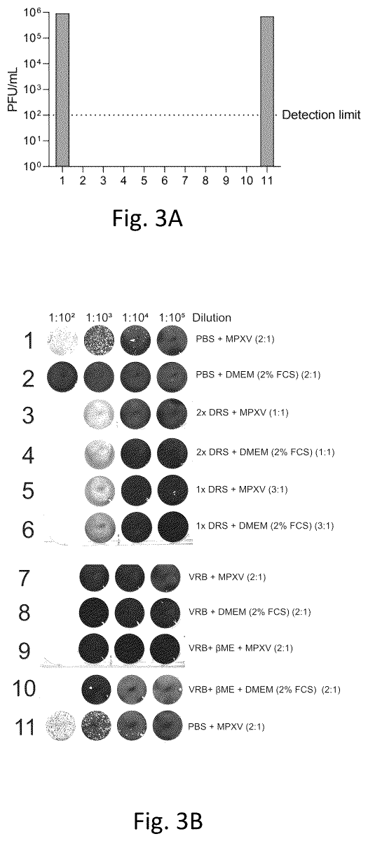

FIG. 4 shows that average Ct-values were consistent when stored at 4° C. according to an exemplary embodiment of the disclosed subject matter.

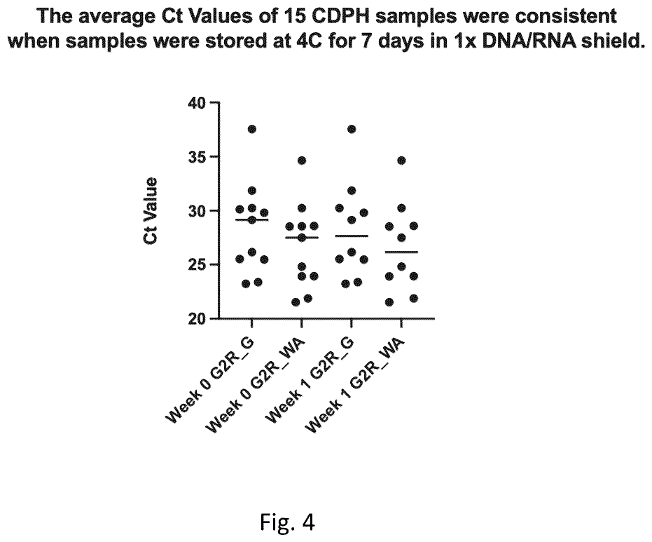

### DETAILED DESCRIPTION

In an effort to bring treatment to people, we developed a highly-sensitive, dual-input multiplex Real Time Reverse Transcription Polymerase Chain Reaction Assay (mRT-PCR). The assay comprises dual inputs comprising viral DNA and mRNA inputs. The assay can detect multiple viral targets, and a human specimen control. The PCR assay performs reverse transcription from mRNA to complementary DNA (cDNA). Real-time amplification of DNA is observed via fluorescence on a thermocycler.

The assay was designed to detect viral mRNA and viral DNA. Orthopoxviruses produce their own RNA once inside the host cell, and packaged virus contains double-stranded DNA (dsDNA). Leveraging this, we found a highly sensitive assay capable of detecting less than 75 copies per mL or 2 copies per assay.

Briefly, in some embodiments, three viral target primers are included in the assay. The first is G2R_G—a generic orthopoxvirus primer probe set. The additional primer and probes detect clade specific regions, allowing for the determination of West African monkeypox virus or Congo Basin monkeypox virus.

In other embodiments, the assay includes primers and probes for RNAS P and two targets that are virus-specific, targeting two monkeypox conservative areas of the genome. Selected mRNA targets include those that are highly selective and highly conserved for the monkeypox virus. They are also highly expressed within the first few days of the lifecycle of the virus to maximize sensitivity in early stages of the disease infection. The virus-specific targeting probes may be allocated within DNA-binding phosphoprotein (2) (I3L) and Protein F11 (F11L) genes of the monkeypox virus. These embodiments of the assay can reduce false negatives compared to embodiments with clade-specific monkeypox targets. These embodiments may also be more robust in detecting monkeypox virus that has evolved beyond the West African or Congo Basin clades.

The assay is high throughput, able to process 90 samples in 2.5 hours and is easily scalable. The assay leverages mRNA and DNA to provide 10× more sensitive than qPCR alone. Because of its sensitivity it can detect virus earlier in infection. This early detection allows for TPOXX® treatment or vaccination as post-exposure prophylaxis.

We saw a need for improved sensitivity in detecting MPXV in many types of patient samples. We improved viral detection in lesion samples using qPCR and mRT-PCR+ techniques.

### EXAMPLES

### Example 1: Introduction of DNAse Treatment to Determine Presence of mRNA Versus DNA Only

Samples delivered to the laboratory are neutralized prior to admission in the lab utilizing 1× or 2×DNA Shield Reagent (Zymo). Liquid samples will be diluted 1:1 in 2×DNA Shield to ensure proper virus neutralization.

Following neutralization and receipt of samples by the laboratory, total DNA and RNA from each patient's sample type is prepared using total Zymo Magnetic Bead RNA/DNA extraction kits on the Kingfisher (thermofisher). Nucleic acids from each sample are then be subjected to the following conditions for 10 minutes at room temperature (100 U/uL) and heat inactivated at 65° C. for 15 minutes:
(1) Non-treated
(2) DNase I treated (removes DNA)
(3) RNase treated (removes RNA)
(4) Treated with both DNAse I and RNAse I

Samples are analyzed by mRT-PCR+, qPCR only, or mRT-PCR only.

A standard operating procedure for the laboratory developed test (LDT) procedure for performing Total RNA/DNA Isolation and multi-target qPCR of DNA from Monkeypoxvirus (MPXV) Samples discussed below.

The purpose of the SOP is to describe the methodology needed to perform qPCR on total DNA/RNA from Monkeypoxvirus (MPXV) positive samples.

### Scope

This SOP covers the detailed steps involved in sample acquisition and testing procedures for the molecular detection of four MPVX specific targets (G2R_G (Generic), G2R_WA (West Africa), C3L (Congo), and RNAse P, allowing Clade-specific detection) in clinical samples.

### General

### Definitions

MPXV: Monkeypox Virus

LDT: Laboratory Developed Test

### Material and Equipment

### Materials

Transport containers

Specimen collection bags and triple packaging

Coolers and cold packs or dry ice

Labels and permanent markers

PPE: disposable anti fluid gown, latex gloves, goggles or full-face cover, lab hat, and shoe covers, and for the elimination of used PPE.

Materials for decontamination of surfaces.

water

TaqPath qRT-PCR master mix

Forward primers

Reverse primers

FAM, HEX, and Cy5 probe

### Equipment

QuantStudio 6 Real-Time PCR System

### General Safety

MPXV PPE Sequence

Monkeypox Virus Testing HazCom Info Sheet

CHEMICAL SAFETY: SAF-003—Laboratory Chemical Hygiene Plan v.1.0.

Use of adequate standard operating procedures (SOPs) must be ensured, and laboratory personnel must be trained for appropriate use of personal protective equipment (PPE) including disposable anti fluid gown, latex gloves, goggles or full-face cover, lab hat, and shoe covers, and for the elimination of used PPE.

Staff should be appropriately trained for specimen collection, storage, packaging, and transport.

Measures should be taken to minimize the risk of laboratory transmission based on a risk assessment at institutional level when testing routine clinical specimens from confirmed or suspected monkeypox patients. These may include limiting the number of staff testing specimens only to staff with proven competency, wearing appropriate PPE, using rigorously applied standard precautions, using effective disinfectants (which include quaternary ammonium compounds and 0.5% (or 200 ppm) bleach (0.5%) on surfaces, and avoiding any procedures that could generate aerosols.

Rigorous adherence to infection prevention and control guidelines must be ensured during specimen collection and handling.

It is recommended that all manipulations of specimens originating from suspected, probable or confirmed cases of monkeypox in the laboratory be conducted according to a risk-based approach. Each laboratory should conduct an institutional risk assessment.

When manipulating biological specimens, core biosafety requirements, similar to those previously referred to as biosafety level 2, must be met and heightened control measures should be applied based on local risk assessment.

Heightened biosafety measures are recommended in addition to the core requirements, including the following for the purpose of clinical testing without virus propagation:

Specimens from patients with suspected MPXV infection must be handled in a reviewed (according to the PAHO laboratory maintenance manual), or certified Class II biosafety cabinet, prior to sample inactivation. Properly inactivated specimens do not require a biosafety cabinet.

Laboratory personnel should wear appropriate PPE, especially for handling specimens before inactivation.

Where use of a centrifuge is required for a procedure, safety cups or sealed rotors should be used.

Additional control measures should be considered for specific procedures, including aerosol-generating procedures, according to the local risk assessment.

### Procedure

### Sample Types

Lesion swabs from patients with suspected Monkeypox virus are resuspended in 1 mL of 1× DNA shield, and 300 uL are used in Kingfisher extractions.

In testing alternative sample types, different neutralizing solutions must be used, according to the table 1 below.

TABLE 1

| Proposed | | |
|---|---|---|
| Sample Type | Reagent | Volume |
| Saliva | 2x DNA/RNA Shield | 1:1 ratio for a final concentration |
| | | of 1x DNA/RNA Shield. |
| Blood, Serum, | 2x DNA/RNA Shield | 1:1 ratio for a final concentration |
| Plasma | | of 1x DNA/RNA shield. |
| Oropharyngeal Swab | 1x DNA/RNA Shield | 1 mL |
| Lesion Swab | 1x DNA/RNA Shield | 1 mL |
| Urine | 2x DNA/RNA Shield | 1:1 ratio for a final concentration |
| | | of 1x DNA/RNA shield |

Perform KingfisherDNA/RNA extraction (R&D-SOP-001, V.1.0.)

Prepare all Wash, Ethanol, and Elution plates as stated in the above referenced SOP.

Place 55 uL of DNA/RNAse free Water in Elution plate.

Establish Molecular Assays on total RNA and DNA samples.

Thaw qRT-PCR TaqPath reagents (Cat #A 15300 or A15299).

Thaw primer/probe mixes.

Set up the following qRT-PCR reaction shown in Table 2.

TABLE 2

| | | Volume per | n = 500 | Final |
|---|---|---|---|---|
| Reagent | Concentration | Reaction | reactions | Concentration |
| TaqPath Master Mix | 4x | 5 uL | 2.5 mL | 1x |
| G2R_G Forward Primer | 100 uM | 0.08 uL | 40 uL | 400 nM |
| G2R_G Reverse Primer | 100 uM | 0.08 uL | 40 uL | 400 nM |
| G2R_WA Forward | 100 uM | 0.08 uL | 40 uL | 400 nM |
| Primer | | | | |
| G2R_WA Reverse | 100 uM | 0.08 uL | 40 uL | 400 nM |
| RNAse P Forward | 100 uM | 0.08 uL | 40 uL | 400 nM |
| Primer | | | | |
| RNAse P Reverse | 100 uM | 0.08 uL | 40 uL | 400 nM |
| Primer | | | | |
| G2R_G-FAM Probe | 100 uM | 0.04 uL | 20 uL | 200 nM |
| G2R_WA HEX Probe | 100 uM | 0.04 uL | 20 uL | 200 nM |
| RNase P-Cy5 Probe | 100 uM | 0.04 uL | 20 uL | 200 nM |
| DNAse/RNAse free | | 9.4 uL | 4.7 mL | |
| Water | | | | |
| | | 15 uL | 7.5 mL | 1x |

Following addition of 15 uL of 1× Master Mix to each well, add 5 uL of each purified DNA/RNA sample. The total reaction volume is 20 uL.

Cover plate and add to Machine, briefly centrifuge, aligning A1 well to A1 plate marker.

Thermocycler Protocol is shown in Table 3.

TABLE 3

| Temperature | Hold time | Number of Cycles |
|---|---|---|
| 25° C. | 2 minutes | x1 |
| 50° C. | 15 minutes | x1 |
| 95° C. | 2 minutes | x1 |
| 95° C. | 3 seconds | |
| 60° C. | 30 seconds | x40* Fluorescence Acquired at this step |

Baseline is set to “AUTO”

Cutoffs are set to 0.05 for each target.

Run assay, and interpret results

Results Interpretation is shown in Table 4.

TABLE 4

| G2R_G | G2R_WA | RNaseP | Result Interpretation |
|---|---|---|---|
| <=38 | >38 or neg | <=38 | MPXV Detected |
| <=38 | <=38 | any | MPXV, West African Clade Detected |
| neg | neg | <=38 | MPXV Not Detected |
| <=38 | >38 or neg | >38 or neg | repeat, consult with supervisor/director |
| neg | neg | >38 | repeat, if same, result as Inconclusive |
| >38 or neg | any | any | repeat, consult with supervisor/director, |
| | | | if still >38, result |

According to the International Health Regulations (IHR), all monkeypox confirmed cases should be notified within 24 hours through official IHR channels.

### Annex

Primers and probes sequences:

G2R_G assay (MPXV generic detection)
G2R_G forward primer
(SEQ ID NO: 1)
5′-GGAAAATGTAAAGACAACGAATACAG
G2R_G reverse primer
(SEQ ID NO: 2)
5′-GCTATCACATAATCTGGAAGCGTA
G2R_G probe
(SEQ ID NO: 3)
5′FAM-AAGCCGTAA/ZEN/TCTATGTTGTCTATCGTGTCC-3′IABKFQ
G2_WA assay (detection of Western African clade viruses)
G2R_WA forward primer
(SEQ ID NO: 4)
5′-CACACCGTCTCTTCCACAGA
G2R_WA reverse primer
(SEQ ID NO: 5)
5′-GATACAGGTTAATTTCCACATCG
G2R_WA probe
(SEQ ID NO: 6)
5′HEX-AACCCGTCGTAACCAGCAATACATTT-3′IABKFQ
Human RNase P (RP)
https://www.ncbi.nlm.nih.gov/pmc/articles/PMC8669853/
RP-F Human RNAse P gene Forward primer
(SEQ ID NO: 7)
AGATTTGGACCTGCGAGCG
RP-R Human RNAse P gene Reverse primer
(SEQ ID NO: 8)
GAGCGGCTGTCTCCACAAGT
RP-P Human RNAse P gene Probe
(SEQ ID NO: 9)
5′Cy5-TTCTGACCT/ZEN/GAAGGCTCTGCGCG-3′IABKFQ

### Validation Procedure

The purpose of this validation is to document the experimental plan and execution of studies needed to validate the qPCR and RT-PCR multiplexed monkeypox (orthopoxvirus) assays disclosed herein for patients suspected of containing monkeypox virus. Patient sample testing will be conducted under CAP/CLIA guidelines. The validation plan includes clinical samples provided by CDPH/UCSF in DNA/RNA Shield to immediately inactivate any infectious agents. Nucleic acid is isolated using the ThermoFisher KingFisher automated extraction and subjected to multiplexed PCR on the QuantStudion 6. Primer/probe combinations detected generic MPXV (G2_G) and specific detection for West African Clade specific MPXV (G2 WA). Primer/probe combinations for specific detection of Congo Basin Clade (C3L) will be utilized in subsequent supplemental validation studies.

### Plan

This plan will cover key analytical parameters including accuracy, precision, reportable range, analytical sensitivity (LOD), analytical specificity, reference interval, specimen stability, reagent stability, carryover, and cross-contamination to comply with CAP/CLIA requirements.

This validation covers lesion fluid collected on cotton/dacron swabs immediately placed in DNA/RNA Shield prior to receipt by the testing laboratory. DNA Shield is a DNA transport and storage medium for any biological sample. DNA/RNA Shield preserves the genetic integrity and expression profiles of samples at ambient temperatures and completely inactivates infectious agents (viruses, bacteria, fungi, & parasites).

### Accuracy

Definition: Accuracy is determined by comparing results to a definitive or reference method, or an established comparative method. For a quantitative test, accuracy refers to ‘closeness to true’ whereas for a qualitative test it refers to correlation to a comparative test or tests that are used to establish ‘true’.

Design: Three different PCR configurations were tested to compare qPCR single-plexed, RT-PCR single-plexed, and mqRT-PCR+ multiplex reactions. Fifteen clinical samples were provided by CDPH/UCSF containing 4 negatives and 11 positive patient samples in DNA/RNA Shield. Experiments were performed with 2 operators.

Experiment 1—qPCR single-plex reactions performed by operator #1 and operator #2.

Experiment 2—RT-PCR single-plex reactions performed by operator #1

Experiment 3—RT-PCR multiplex reactions performed by operator #1 & #2 DATA

Experiment 1: Concordance run of Single-plex primers. All samples were purified by Kingfisher using the total DNA/RNA Extraction Kit by Zymo. A total of 2 uL of template was amplified in this reaction by multiple operators.

Table 5 shows the Experiment 1 Kingfisher Plate Setup (96-well plate). Samples were purified once and used in the single-plex qPCR assay.

TABLE 5

| | 1 | 2 |
|---|---|---|
| A | NTC | Sample 8 |
| B | Sample 1 | Sample 9 |
| C | Sample 2 | Sample 10 |
| D | Sample 3 | Sample 11 |
| E | Sample 4 | Sample 12 |
| F | Sample 5 | Sample 13 |
| G | Sample 6 | Sample 14 |
| H | Sample 7 | Sample 15 |

| | Expected Result |
|---|---|
| Sample | (UCSF/CDPH) |
| 1 | POS WA |
| 2 | POS WA |
| 3 | POS WA |
| 4 | POS WA |
| 5 | POS WA |
| 6 | POS WA |
| 7 | POS WA |
| 8 | NEG |
| 9 | WEAK POS WA |
| 10 | POS WA |
| 11 | NEG |
| 12 | POS WA |
| 13 | POS WA |
| 14 | NEG |
| 15 | NEG |

Table 6 summarizes the qPCR setup for singleplex qPCR. Singleplex qPCR was performed on 2 uL of samples extracted by Kingfisher. The reagents are detailed below in Table 6. Table 7 shows the qPCR settings for the thermocycler protocol for singleplex qPCR.

TABLE 6

| | Volume per | Final |
|---|---|---|
| Reagent | Reaction (n) | Concentration |
| Taqman Fast Advanced Master Mix (2x) | 10 uL | 1x |
| Singleplex Primer/Probe Mix (40x); G2R_G; | 1 uL | 2x |
| G2R_WA; C3L; or RNAseP pre-mixes. | | |
| DNAse/RNAse-free water | 7 uL | |
| Sample | 2 uL | |
| Total | 20 uL | |

TABLE 7

| Temperature | Hold time | Number of Cycles |
|---|---|---|
| 95° C. | 20 seconds | x1 |
| 95° C. | 1 second | |
| 60° C. | 20 seconds | x40 |

Tables 8 and 9 show singleplex qPCR results from multiple operators on the same machine. Samples were run and data are below.

TABLE 8

| qPCR-1 | Operator #1 | | | | | |
|---|---|---|---|---|---|---|
| Sample | G2R_G | G2R_WA | C3L | RNAse P | RB Result | Expected Result |
| 1 | 30.673 | 28.482 | Undetermined | NA | POS WA | POS WA |
| 2 | 32.512 | 30.397 | Undetermined | NA | POS WA | POS WA |
| 3 | 30.947 | 28.587 | Undetermined | NA | POS WA | POS WA |
| 4 | 25.996 | 23.867 | Undetermined | NA | POS WA | POS WA |
| 5 | 29.804 | 27.668 | Undetermined | NA | POS WA | POS WA |
| 6 | 30.256 | 28.427 | Undetermined | NA | POS WA | POS WA |
| 7 | 23.690 | 21.886 | Undetermined | NA | POS WA | POS WA |
| 8 | Undetermined | Undetermined | Undetermined | NA | NEG | NEG |
| 9 | 37.219 | Undetermined | Undetermined | NA | WEAK POS | WEAK POS WA |
| 10 | 26.702 | 24.999 | Undetermined | NA | POS WA | POS WA |
| 11 | Undetermined | Undetermined | Undetermined | NA | NEG | NEG |
| 12 | 26.152 | 23.976 | Undetermined | NA | POS WA | POS WA |
| 13 | 23.603 | 21.486 | Undetermined | NA | POS WA | POS WA |
| 14 | Undetermined | Undetermined | Undetermined | NA | NEG | NEG |
| 15 | Undetermined | Undetermined | Undetermined | NA | NEG | NEG |

TABLE 9

| | qPCR2 | Operator #2 | | | | |
|---|---|---|---|---|---|---|
| | G2R_G | G2R_WA | C3L | RNAse P | RB Result | Expected Result |
| 1 | 29.587 | 28.558 | Undetermined | 36.027 | POS WA | POS WA |
| 2 | 31.190 | 30.099 | Undetermined | 35.589 | POS WA | POS WA |
| 3 | 29.537 | 28.534 | Undetermined | Undetermined | POS WA | POS WA |
| 4 | 25.016 | 23.938 | Undetermined | 33.734 | POS WA | POS WA |
| 5 | 28.437 | 27.269 | Undetermined | 35.650 | POS WA | POS WA |
| 6 | 29.351 | 28.581 | Undetermined | Undetermined | POS WA | POS WA |
| 7 | 23.049 | 21.869 | Undetermined | 31.229 | POS WA | POS WA |
| 8 | Undetermined | Undetermined | Undetermined | 31.232 | NEG | NEG |
| 9 | 37.871 | 34.647 | Undetermined | Undetermined | WEAK POS WA | WEAK POS WA |
| 10 | 25.568 | 24.623 | Undetermined | 34.135 | POS WA | POS WA |
| 11 | Undetermined | Undetermined | Undetermined | Undetermined | NEG | NEG |
| 12 | 24.771 | 23.872 | Undetermined | 34.159 | POS WA | POS WA |
| 13 | 22.849 | 21.563 | Undetermined | 33.662 | POS WA | POS WA |
| 14 | Undetermined | Undetermined | Undetermined | 31.797 | NEG | NEG |
| 15 | Undetermined | Undetermined | Undetermined | 31.243 | NEG | NEG |

Experiment 2: Single-plex amplification by RT-PCR. All samples were purified by Kingfisher using the total DNA/RNA Extraction Kit by Zymo. A total of 2 uL of template was amplified in this reaction by multiple operators.

TABLE 10
Sample layout for Kingfisher Extractions.

| | 1 | 2 |
|---|---|---|
| A | NTC | Sample 8 |
| B | Sample 1 | Sample 9 |
| C | Sample 2 | Sample 10 |
| D | Sample 3 | Sample 11 |
| E | Sample 4 | Sample 12 |
| F | Sample 5 | Sample 13 |
| G | Sample 6 | Sample 14 |
| H | Sample 7 | Sample 15 |

Tables 11 through 13 summarize a qPCR setup for singleplex qPCR Singleplex qPCR was performed on 2 uL of samples extracted by Kingfisher. The reagents are detailed below in Table 11. Table 12 shows the qPCR Settings of the thermocycler protocol for singleplex qPCR. Table 13 shows data generated from RT-PCR using total DNA/RNA as input.

TABLE 11

| | Volume per | Final |
|---|---|---|
| Reagent | Reaction (n) | Concentration |
| Taqpath Master Mix (4x) | 5 uL | 1x |
| Singleplex Primer/Probe Mix | 1 uL | 2x |
| (40x); G2R_G; G2R_WA; C3L; | | |
| or RNAseP pre-mixes. | | |
| DNAse/RNAse-free H2O | 12 uL | |
| Sample | 2 uL | |
| Total | 20 uL | |

TABLE 12

| Temperature | Hold time | Number of Cycles |
|---|---|---|
| 25° C. | 2 minutes | x1 |
| 50° C. | 15 minutes | x1 |
| 95° C. | 2 minutes | x1 |
| 95° C. | 3 seconds | |
| 60° C. | 30 seconds | x40 |

TABLE 13

| | gRT-PCR 1 | Operator #2 | | | | |
|---|---|---|---|---|---|---|
| | G2R_G | G2R_WA | C3L | RNAse P | RB Result | Expected Result |
| 1 | 27.549 | 27.890 | Undetermined | 36.341 | POS WA | POS WA |
| 2 | 28.966 | 29.459 | Undetermined | 35.629 | POS WA | POS WA |
| 3 | 27.584 | 27.954 | Undetermined | 38.181 | POS WA | POS WA |
| 4 | 22.892 | 23.344 | Undetermined | 32.675 | POS WA | POS WA |
| 5 | 26.527 | 26.771 | Undetermined | 35.426 | POS WA | POS WA |
| 6 | 25.302 | 26.189 | Undetermined | 38.367 | POS WA | POS WA |
| 7 | 21.273 | 21.298 | Undetermined | 30.888 | POS WA | POS WA |
| 8 | Undetermined | Undetermined | Undetermined | 31.026 | NEG | NEG |
| 9 | 34.583 | 34.500 | Undetermined | Undetermined | POS WA | WEAK POS WA |
| 10 | 24.348 | 24.367 | Undetermined | 34.078 | POS WA | POS WA |
| 11 | Undetermined | Undetermined | Undetermined | Undetermined | NEG | NEG |
| 12 | 23.487 | 23.482 | Undetermined | 34.995 | POS WA | POS WA |
| 13 | 21.479 | 21.422 | Undetermined | 34.797 | POS WA | POS WA |
| 14 | Undetermined | Undetermined | Undetermined | 32.302 | NEG | NEG |
| 15 | Undetermined | Undetermined | Undetermined | 31.472 | NEG | NEG |

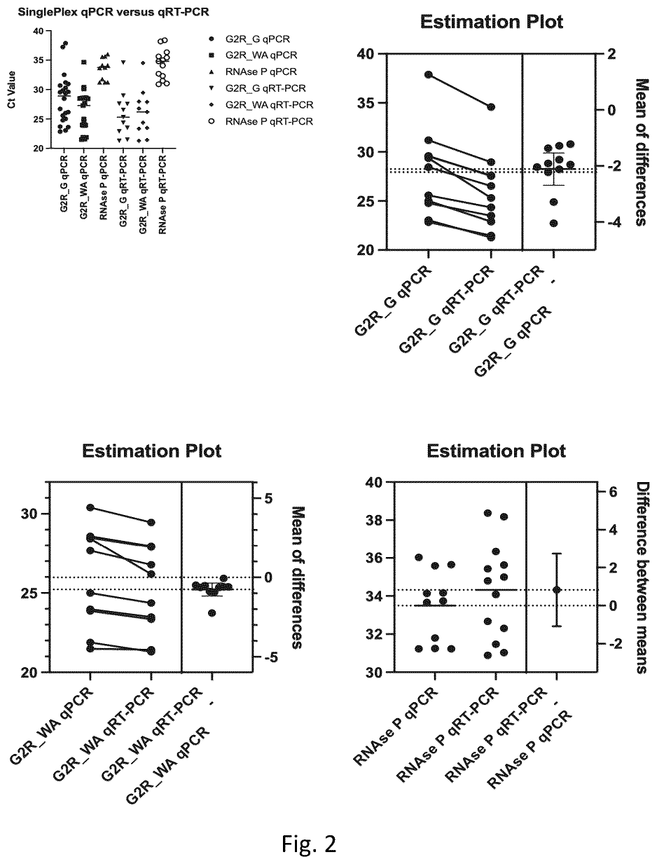

FIG. 2 shows the Average Difference in Ct-values based on method, qPCR versus RT-PCR Single-plex data indicate that there is on average an 8-fold increase of viral nucleic acid template as detected by G2R_G in the RT-PCR assay.

Experiment 3: Dual input (DNA/RNA) multiplex Reverse Transcription-PCR (mRT-PCR+) assessment: All samples were processed by Kingfisher according to the R&D SOP using the Total DNA/RNA Zymo kit. The Sample layout is detailed below. Following extraction, 5 uL of sample was included in the mRT-PCR+, and two operators performed the experiment.

Table 14 shows the layout of Kingfisher Sample Extraction for DNA/RNA Isolation using the Zymo Kit. This extraction was performed on clinical samples and diluted positive control (G2R template, 125 bp) for LoD studies. Table 15 shows the mRT-PCR+ recipe.

TABLE 14
Kingfisher Sample Plate

| | 1 | 2 | 3 | 4 |
|---|---|---|---|---|
| A | NTC | Sample 8 | 2.00E−01 | 2.00E−09 |
| B | Sample 1 | Sample 9 | 2.00E−02 | 2.00E−10 |
| C | Sample 2 | Sample 10 | 2.00E−03 | NTC |
| D | Sample 3 | Sample 11 | 2.00E−04 | NTC |
| E | Sample 4 | Sample 12 | 2.00E−05 | NTC |
| F | Sample 5 | Sample 13 | 2.00E−06 | NTC |
| G | Sample 6 | Sample 14 | 2.00E−07 | NTC |
| H | Sample 7 | Sample 15 | 2.00E−08 | NTC |

TABLE 15

| | | Volume | | Final |
|---|---|---|---|---|
| | Con- | per | n = 500 | Con- |
| Reagent | centration | Reaction | reactions | centration |
| TaqPath Master Mix | 4x | 5 uL | 2.5 mL | 1x |
| G2R_G Forward Primer | 100 uM | 0.08 uL | 40 uL | 400 nM |
| G2R_G Reverse Primer | 100 uM | 0.08 uL | 40 uL | 400 nM |
| G2R_WA Forward | 100 uM | 0.08 uL | 40 uL | 400 nM |
| Primer | | | | |
| G2R_WA Reverse | 100 uM | 0.08 uL | 40 uL | 400 nM |
| RNAse P Forward | 100 uM | 0.08 uL | 40 uL | 400 nM |
| Primer | | | | |
| RNAse P Reverse | 100 uM | 0.08 uL | 40 uL | 400 nM |
| Primer | | | | |
| G2R_G-FAM Probe | 100 uM | 0.04 uL | 20 uL | 200 nM |
| G2R_WA HEX Probe | 100 uM | 0.04 uL | 20 uL | 200 nM |
| RNase P-Cy5 Probe | 100 uM | 0.04 uL | 20 uL | 200 nM |
| DNAse/RNAse free | | 9.4 uL | 4.7 mL | |
| Water | | | | |
| | | 15 uL | 7.5 mL | 1x |

Following addition of 15 uL of 1× Master Mix to each well, add 5 uL of each purified DNA/RNA sample. The total reaction volume is 20 uL.

Table 16 shows the mRT-PCR+96-well plate setup on Fast Plates. This includes samples and positive control LoD.

TABLE 16

| | 1 | 2 | 3 | 4 | 5 | 6 | 7 | 8 | 9 | 10 | 11 | 12 |
|---|---|---|---|---|---|---|---|---|---|---|---|---|
| A | NTC | NTC | NTC | Sample | Sample | Sample | 2.00E−01 | 2.00E−01 | 2.00E−01 | 2.00E−09 | 2.00E−09 | 2.00E−09 |
| | | | | 8 | 8 | 8 | | | | | | |
| B | Sample | Sample | Sample | Sample | Sample | Sample | 2.00E−02 | 2.00E−02 | 2.00E−02 | 2.00E−10 | 2.00E−10 | 2.00E−10 |
| | 1 | 1 | 1 | 9 | 9 | 9 | | | | | | |
| C | Sample | Sample | Sample | Sample | Sample | Sample | 2.00E−03 | 2.00E−03 | 2.00E−03 | NTC | NTC | NTC |
| | 2 | 2 | 2 | 10 | 10 | 10 | | | | | | |
| D | Sample | Sample | Sample | Sample | Sample | Sample | 2.00E−04 | 2.00E−04 | 2.00E−04 | NTC | NTC | NTC |
| | 3 | 3 | 3 | 11 | 11 | 11 | | | | | | |
| E | Sample | Sample | Sample | Sample | Sample | Sample | 2.00E−05 | 2.00E−05 | 2.00E−05 | NTC | NTC | NTC |
| | 4 | 4 | 4 | 12 | 12 | 12 | | | | | | |
| F | Sample | Sample | Sample | Sample | Sample | Sample | 2.00E−06 | 2.00E−06 | 2.00E−06 | NTC | NTC | NTC |
| | 5 | 5 | 5 | 13 | 13 | 13 | | | | | | |
| G | Sample | Sample | Sample | Sample | Sample | Sample | 2.00E−07 | 2.00E−07 | 2.00E−07 | NTC | NTC | NTC |
| | 6 | 6 | 6 | 14 | 14 | 14 | | | | | | |
| H | Sample | Sample | Sample | Sample | Sample | Sample | 2.00E−08 | 2.00E−08 | 2.00E−08 | NTC | NTC | NTC |
| | 7 | 7 | 7 | 15 | 15 | 15 | | | | | | |

TABLE 17

| | | G2R_G | G2R_WA | Rnase P | G2R_G | G2R_WA | Rnase P |
|---|---|---|---|---|---|---|---|
| Sample | Replicate | (Operator #1) | (Operator #1) | (Operator #1) | (Operator #2) | (Operator #2) | (Operator #2) |
| 1 | a | 25.833 | 27.126 | 30.727 | 26.683 | 27.271 | 32.537 |
| | b | 25.825 | 27.102 | 30.345 | 26.810 | 27.429 | 32.856 |
| | c | 25.663 | 27.137 | 30.608 | 26.742 | 27.456 | 32.577 |
| 2 | a | 27.604 | 29.096 | 30.393 | 28.395 | 29.401 | 32.967 |
| | b | 27.533 | 28.866 | 29.934 | 28.510 | 29.306 | 32.726 |
| | c | 27.334 | 28.896 | 30.221 | 28.438 | 29.338 | 32.658 |
| 3 | a | 25.870 | 27.246 | 32.959 | 26.877 | 27.511 | 34.568 |
| | b | 25.874 | 27.296 | 34.267 | 26.891 | 27.568 | 35.799 |
| | c | 25.838 | 27.339 | 34.235 | 26.829 | 27.537 | 34.770 |
| 4 | a | 21.573 | 22.856 | 28.693 | 22.366 | 23.093 | 30.939 |
| | b | 21.600 | 22.926 | 28.576 | 22.515 | 23.188 | 30.824 |
| | c | 21.541 | 22.944 | 28.408 | 22.433 | 23.166 | 30.830 |
| 5 | a | 25.383 | 26.555 | 30.656 | 26.205 | 26.817 | 33.766 |
| | b | 25.317 | 26.576 | 31.428 | 26.310 | 26.850 | 33.318 |
| | c | 25.300 | 26.595 | 30.996 | 26.114 | 26.803 | 33.631 |
| 6 | a | 23.463 | 25.236 | 36.185 | 24.252 | 25.560 | 36.167 |
| | b | 23.442 | 25.245 | 35.209 | 24.265 | 25.534 | 37.856 |
| | c | 23.414 | 25.283 | 34.997 | 24.236 | 25.649 | 36.806 |
| 7 | a | 19.661 | 20.829 | 26.751 | 20.441 | 21.005 | 28.973 |
| | b | 19.617 | 20.897 | 26.480 | 20.486 | 21.137 | 28.884 |
| | c | 19.747 | 21.042 | 26.812 | 20.470 | 21.121 | 28.907 |
| 8 | a | 36.105 | Undetermined | 25.913 | Undetermined | Undetermined | 28.857 |
| | b | Undetermined | Undetermined | 31.620 | Undetermined | Undetermined | 28.914 |
| | c | Undetermined | Undetermined | 25.943 | Undetermined | Undetermined | 28.719 |
| 9 | a | 31.966 | 33.142 | Undetermined | 32.567 | 34.014 | Undetermined |
| | b | 31.806 | 33.419 | Undetermined | 32.388 | 33.266 | Undetermined |
| | c | 31.584 | 33.129 | Undetermined | 32.599 | 33.323 | Undetermined |
| 10 | a | 22.013 | 23.415 | 29.493 | 22.927 | 23.653 | 31.746 |
| | b | 21.976 | 23.363 | 29.520 | 22.934 | 23.638 | 31.993 |
| | c | 21.926 | 23.352 | 29.388 | 22.910 | 23.614 | 31.650 |
| 11 | a | Undetermined | Undetermined | 35.478 | Undetermined | Undetermined | Undetermined |
| | b | Undetermined | Undetermined | Undetermined | Undetermined | Undetermined | 37.138 |
| | c | Undetermined | Undetermined | 35.200 | Undetermined | Undetermined | Undetermined |
| 12 | a | 21.381 | 22.786 | 29.185 | 22.310 | 23.131 | 31.468 |
| | b | 21.464 | 22.862 | 29.190 | 22.412 | 23.191 | 31.573 |
| | c | 21.410 | 22.761 | 29.218 | 22.292 | 23.072 | 31.408 |
| 13 | a | 18.986 | 20.323 | 28.481 | 19.887 | 20.604 | 30.736 |
| | b | 18.918 | 20.334 | 28.429 | 19.822 | 20.573 | 30.652 |
| | c | 18.958 | 20.306 | 28.452 | 19.827 | 20.569 | 30.744 |
| 14 | a | Undetermined | Undetermined | 26.433 | Undetermined | Undetermined | 29.340 |
| | b | Undetermined | Undetermined | 26.472 | Undetermined | Undetermined | 29.241 |
| | c | Undetermined | Undetermined | 26.532 | Undetermined | Undetermined | 29.161 |
| 15 | a | Undetermined | Undetermined | 26.162 | Undetermined | Undetermined | 28.813 |
| | b | Undetermined | Undetermined | 26.011 | Undetermined | Undetermined | 28.862 |
| | c | Undetermined | Undetermined | 25.969 | Undetermined | Undetermined | 28.795 |

Accuracy Conclusion. Utilizing 15 clinical samples run in triplicate across 2 different operators showed an average difference in Ct-values based on method. qPCR versus RT-PCR Single-plex data indicate that there is on average an 8-fold increase of viral nucleic acid template as detected by G2R_G in the RT-PCR assay. Data above shows 99% concordance with results obtained by CDPH/UCSF over multiple runs with multiple operators. The only discordant result was a single reaction from a triplicate showing low level contamination that would not have been reported out as discordant.

### Analytical Precision/Reproducibility

Definition: Records for validation of precision must show that a test will return the same result regardless of minor variations in testing conditions that can cause random error, such as different technologists, instruments, reagent lots, days, etc. This is usually determined by repeated measures of samples throughout the reportable range.

Design: Reproducibility (across-site precision) data was obtained by utilizing 2 operators, across 2 days with 2 unique PCR runs. A second reproducibility experiment was conducted with 2 operators across 2 QuantStudio™ 6 instruments. Repeatability (within-site precision) data was obtained by running 15 patient samples in triplicate on the same PCR plate/run.

Table 18 shows data from two operators of the mRT-PCR+ Assay run in triplicate. Average and Standard Deviation between operators' columns are shown. Detailed data displayed in Table 17 above.

TABLE 18

| | G2R_G | G2R_WA | Rnase P | G2R_G | G2R_WA | Rnase P |
|---|---|---|---|---|---|---|
| Sample | (OP 1) | (OP 1) | (OP 1) | (OP 2) | (OP 2) | (OP 2) |
| 1 | 25.7 | 27.1 | 30.6 | 26.7 | 27.5 | 32.6 |
| 2 | 27.3 | 28.9 | 30.2 | 28.4 | 29.3 | 32.7 |
| 3 | 25.8 | 27.3 | 34.2 | 26.8 | 27.5 | 34.8 |
| 4 | 21.5 | 22.9 | 28.4 | 22.4 | 23.2 | 30.8 |
| 5 | 25.3 | 26.6 | 31.0 | 26.1 | 26.8 | 33.6 |
| 6 | 23.4 | 25.3 | 35.0 | 24.2 | 25.6 | 36.8 |
| 7 | 19.7 | 21.0 | 26.8 | 20.5 | 21.1 | 28.9 |
| 8 | Undetermined | Undetermined | 25.9 | Undetermined | Undetermined | 28.7 |
| 9 | 31.6 | 33.1 | Undetermined | 32.6 | 33.3 | Undetermined |
| 10 | 21.9 | 23.4 | 29.4 | 22.9 | 23.6 | 31.7 |
| 11 | Undetermined | Undetermined | 35.2 | Undetermined | Undetermined | Undetermined |
| 12 | 21.4 | 22.8 | 29.2 | 22.3 | 23.1 | 31.4 |
| 13 | 19.0 | 20.3 | 28.5 | 19.8 | 20.6 | 30.7 |
| 14 | Undetermined | Undetermined | 26.5 | Undetermined | Undetermined | 29.2 |
| 15 | Undetermined | Undetermined | 26.0 | Undetermined | Undetermined | 28.8 |
| | G2R_G | G2R_G | G2R_WA | G2R_WA | Rnase P | Rnase P |
| Sample | Average | SD | Avg | SD | Avg | SD |
| 1 | 26.3 | 0.5 | 27.3 | 0.2 | 31.6 | 1.2 |
| 2 | 28.0 | 0.5 | 29.2 | 0.2 | 31.5 | 1.4 |
| 3 | 26.4 | 0.6 | 27.4 | 0.1 | 34.4 | 0.9 |
| 4 | 22.0 | 0.5 | 23.0 | 0.1 | 29.7 | 1.3 |
| 5 | 25.8 | 0.5 | 26.7 | 0.1 | 32.3 | 1.4 |
| 6 | 23.8 | 0.4 | 25.4 | 0.2 | 36.2 | 1.1 |
| 7 | 20.1 | 0.4 | 21.0 | 0.1 | 27.8 | 1.2 |
| 8 | | | | | 28.3 | 2.2 |
| 9 | 32.2 | 0.4 | 33.4 | 0.3 | | |
| 10 | 22.4 | 0.5 | 23.5 | 0.1 | 30.6 | 1.3 |
| 11 | | | | | 35.9 | 1.0 |
| 12 | 21.9 | 0.5 | 23.0 | 0.2 | 30.3 | 1.3 |
| 13 | 19.4 | 0.5 | 20.5 | 0.1 | 29.6 | 1.2 |
| 14 | | | | | 27.9 | 1.5 |
| 15 | 39.6 | | | | 27.4 | 1.5 |

Analytical Precision and Reproducibility Conclusion. Utilizing 15 clinical samples run in triplicate across 2 different operators the above data shows standard deviations between 0.1 and 1.4 for positive patient samples. Table 19 below shows LOD studies performed on synthetic control samples by 2 different operators with 2 different instruments (Oak2 & Oak4) showing standard deviations between 0.467 to 0.794 across 70 reactions for G2R_G and G2R_WA. All experiments in table 17 and table 18 were run in triplicate demonstrating within run repeatability. These results indicate very low levels of variability across instruments and across operators with a variety of patient samples and control material.

### Analytical Sensitivity (LOD) and Specificity

Definition. The analytical sensitivity (lower limit of detection) refers to the ability of a test to confidently or consistently detect at the lowest amount of analyte present. Analytical specificity refers to the ability of a test or procedure to correctly identify or quantify an entity in the presence of interfering or cross-reactive substances.

Design. Synthetically derived control material was generated for 2 targets: generic MPXV (G2R_G) and West African Clade (G2R_WA). This control material was diluted in deionized water based on the table below. Dilutions were run in triplicate with 2 different operators and 2 different instruments. No template control (NTC) was also calculated across 70 reactions to determine specificity of amplification in the absence of template. Table 19 summarizes an LOD Dilution Study for Operators #1 and #2. Table 20 summarizes NTC Amplification with multiple primer/probe sets. Table 21 summarizes amplification with multiple primer/probe sets with two operators. Table 21 summarizes amplification with multiple primer/probe sets with 2 operators.

TABLE 19
Operator #2

| | Concentration | Detection | Mean | STD |
|---|---|---|---|---|
| Target | (genomic copies/mL) | Rate | Ct | DEV (Ct) |
| | 1.48E+08 | 3/3 (100%) | 12.416 | 0.0339 |
| | 1.48E+07 | 3/3 (100%) | 15.691 | 0.0398 |
| | 7.42E+07 | 3/3 (100%) | 15.808 | 0.6360 |
| | 1.48E+06 | 3/3 (100%) | 19.115 | 0.0637 |
| | 1.48E+05 | 3/3 (100%) | 22.501 | 0.0344 |
| | 1.48E+04 | 3/3 (100%) | 25.948 | 0.0080 |
| G2R_G | 7.42E+04 | 3/3 (100%) | 24.281 | 0.1420 |
| | 1.48E+03 | 3/3 (100%) | 29.395 | 0.0730 |
| | 1.48E+02 | 3/3 (100%) | 32.123 | 0.2917 |
| | 7.42E+01 | 19/20 (95%) | 34.134 | 0.5615 |
| | 1.48E+01 | 2/3 (67%) | 35.125 | 0.0389 |
| | 1.48E+00 | 1/3 (33%) | UND | UND |
| | 0 | 0/3 (0%) | UND | UND |
| | 1.48E+08 | 3/3 (100%) | 17.939 | 0.1552 |
| | 1.48E+07 | 3/3 (100%) | 21.27 | 0.1951 |
| | 7.42E+07 | 3/3 (100%) | 21.395 | 0.8760 |
| | 1.48E+06 | 3/3 (100%) | 24.808 | 0.2593 |
| | 1.48E+05 | 3/3 (100%) | 28.377 | 0.3025 |
| | 1.48E+04 | 3/3 (100%) | 31.911 | 0.3474 |
| G2R_WA | 7.42E+04 | 3/3 (100%) | 30.129 | 0.2880 |
| | 1.48E+03 | 3/3 (100%) | 35.081 | 0.2793 |
| | 1.48E+02 | 3/3 (100%) | 37.589 | 0.1304 |
| | 7.42E+01 | 3/20 (15%) | 39.059 | UND |
| | 1.48E+01 | 0/3(0%) | UND | UND |
| | 1.48E+00 | 0/3(0%) | UND | UND |

TABLE 20

| Target | Operator | Detection Rate at LoD | Average Ct |
|---|---|---|---|
| G2R_G | 1 | 3/70 (4%) | 35.80 |
| | 2 | 0/70 (0%) | UND |
| G2R_WA | 1 | 0/70 (0%) | UND |
| | 2 | 0/870 (0%) | UND |

TABLE 21

| Oak 2 | Operator #2 | | | | | | LoD | |
|---|---|---|---|---|---|---|---|---|
| Primer/ | Average | 95% CI | | | | | (copies/ | |
| Probe | Ct | SD Ct | Sensitivity | Specificity | PPV | NPV | mL) | Accuracy |
| G2R_G | 34.116 | 0.468 | 96.15% | 100.00% | 100.00% | 98.59% | 74.20 | 98.96% |
| G2R_WA | 39.122 | 0.47127805 | | | | | | |
| Rnase P | und | und | | | | | | |

Analytical Sensitivity and Specificity Conclusion. Tables 20 and 21 above summarize data from prior tables showing analytical sensitivity, analytical specificity, accuracy, and LOD down to 74.2 copies/mL of the viral genome.

### Linearity and Analytical Measurement Range (Amr)

N/A since it is a qualitative test.

### Reportable Range and Reference Intervals

Detected: A non-variola orthopoxvirus was detected. In addition to monkeypox, there are several viral species in the genus orthopoxvirus. But since there are no current epidemiological concerns about those other viruses, a positive result is presumptive positive for monkeypox. Variola is the virus that causes smallpox. This test will not detect smallpox.

Not Detected: This means that an orthopoxvirus was not detected, and the patient is therefore negative for monkeypox.

Equivocal: This result can occur when the virus is detected at levels close to the limit of detection of the assay, and a definitive result cannot be determined. For any equivocal result, the CDC recommends that a new patient sample is collected and tested.

Inconclusive: This result can occur when the assay control criteria are not met and no virus is detected. The concern here is a poorly collected sample, and the CDC recommends that a new patient sample should be collected and tested.

Reportable range of the monkeypox RT-PCR reportable ranges will be “Detected” or “Not Detected.”

### Specificity and Cross Reactivity of Primers—in Silico Analysis

“Analytical specificity” refers to the ability of an assay to measure on particular organism or substance, rather than others, in a sample. An assay's analytical sensitivity and analytical specificity are distinct from that assay's clinical diagnostic sensitivity and diagnostic specificity. “Diagnostic sensitivity” is the percentage of persons who have a given disorder who are identified by the assay as positive for the disorder. High analytical sensitivity does not guarantee acceptable diagnostic sensitivity. “Diagnostic specificity” is the percentage of persons who do not have a given condition who are identified by the assay as negative for the condition. False-positive reactions occur because of sample contamination and diminish the diagnostic specificity of the assay. The terms “sensitivity” and “specificity” should be used with the requisite adjectives because the “diagnostic” and the “analytical” meanings of these terms are very different.

To determine whether or not primers were specific to Monkeypox virus, we performed a nucleotide BLAST on the primer and probe sequences. There were no hits within other orthopoxviruses or other clades.

In silico nBLAST parameters were set to include homologies ranging from 0-100%. Neither Primer nor Probe sequences from G2R_G or G2R_WA sets had homology with other organisms.

Analytical Specificity for the assay is 100%.

### Specimen and Reagent Stability

Design. Data provided by CDPH on inactivation of MPXV live cultures with DNA/RNA shield. Vero E6 cells were seeded 24 h before infection in 12-well plates (4.2×105 cells/well). MPXV stock of a clinical isolate from the Institute of Virologie UKF Freiburg (#360.1.1) was diluted with PBS or the Zymo buffers as indicated and incubated for 30 min at room temperature. The mixtures were then diluted in PBS in four consecutive 1:10 steps from 1:100 to 1:100,000 and 1 ml of the dilutions were added to the cells for one hour at 37° C. Then the inoculum was removed and the cells were overlaid with 1 ml DMEM, 1% FCS and 0.6% Oxoid agar for 4 days, 37° C., 5% C02. Cells were fixed with 3.7% formaldehyde and stained with Crystal violet solution. The dotted line indicates the limit of detection at the 1:100 dilution.

Table 22 Monkeypox virus (MPXV) inactivation with Zymo buffers.

Specimen and reagent stability conclusion. Based on the Zymo DNA/RNA Shield package insert: cells, tissues, blood, plasma, serum, saliva, urine, feces and environmental samples are to be collected in 10% (v/v or w/v). RNA is stable at ambient temperature (4° C. to 25° C.) for >1 month, DNA is stable at ambient temperature (4° C.-25° C.) for >2 years, and DNA & RNA is stable frozen (<−20° C.) indefinitely. Other reagents are stored and stable based on manufacturers' specifications.

At 4° C., we found that samples maintained their integrity for at least 1 week, pre-extraction when resuspended in 1× DNA/RNA Shield which was consistent with CDPH's results, below.

### Carryover and Cross Contamination

Definition. This item is primarily directed at ensuring adequate physical separation of pre- and post-amplification samples to avoid amplicon contamination. The extreme sensitivity of amplification systems requires that the laboratory take special precautions. For example, pre- and post-amplification samples should be manipulated in physically separate areas; gloves must be worn and frequently changed during processing; dedicated pipettes (positive displacement type or with aerosol barrier tips) must be used; and manipulations must minimize aerosolization.

Design. The laboratory has physical separation between pre- and post-amplification work areas. To determine potential contamination during extraction or during PCR, we designed a “checkerboard” experiment alternating positive and negative samples for nucleic acid isolation and subsequent qmRT-PCR+ for MPXV. Table 19 summarizes the checkerboard extraction and PCR for Monkeypox virus (MPXV).

TABLE 23

| mRT-PCR + Plate | | | | | | | | | | | | |
|---|---|---|---|---|---|---|---|---|---|---|---|---|
| Map | | | Jul. 17, 2022 | | | | | | | | | |
| | 1 | 2 | 3 | 4 | 5 | 6 | 7 | 8 | 9 | 10 | 11 | 12 |
| A | NTC | NTC | NTC | NTC | NTC | NTC | NTC | NTC | NTC | NTC | NTC | NTC |
| B | NTC | 1:1e3 | NTC | 1:1e3 | NTC | 1:1e3 | NTC | 1:1e3 | NTC | NTC | NTC | NTC |
| C | NTC | NTC | 1:1e6 | NTC | 1:1e6 | NTC | NTC | NTC | NTC | NTC | NTC | NTC |
| D | NTC | 1:1e9 | NTC | 1:1e9 | NTC | 1:1e9 | NTC | 1:1e9 | NTC | 1:1e9 | NTC | NTC |
| E | NTC | NTC | 1:1e9 | NTC | 1:1e9 | NTC | 1:1e9 | NTC | 1:1e9 | NTC | 1:1e9 | NTC |
| F | NTC | 1:1e9 | NTC | 1:1e9 | NTC | 1:1e9 | NTC | 1:1e9 | NTC | 1:1e9 | NTC | NTC |
| G | NTC | NTC | 1:1e9 | NTC | 1:1e9 | NTC | 1:1e9 | NTC | 1:1e9 | NTC | 1:1e9 | NTC |
| H | NTC | NTC | NTC | NTC | NTC | NTC | NTC | NTC | NTC | NTC | NTC | NTC |

Carryover and Cross Contamination Conclusion. The Renegade.bio clinical lab is designed for unidirectional workflow and separation of pre- and post-amplification rooms. The above experiment tested any contamination between wells during amplification and PCR. No contamination was detected.

### Assay Principles

There are three targets. The target specific probes are: G2R_G (Generic), G2R_WA (West Africa), and RNAse P, allowing Clade-specific detection. The primer/probe set to detect the human RNase P gene (RP) is included in the panel for extraction control. DNA from patient specimens is amplified on the Applied Biosystem QuantStudio 6 instruments with SDS version 1.3 software.

### Primer, Probe, and Control Sequences

G2R_G assay (MPXV generic detection)
G2R_G forward primer
(SEQ ID NO: 1)
5′-GGAAAATGTAAAGACAACGAATACAG
G2R_G reverse primer
(SEQ ID NO: 2)
5′-GCTATCACATAATCTGGAAGCGTA
G2R_G probe
(SEQ ID NO: 10)
5′FAM-AAGCCGTAATCTATGTTGTCTATCGTGTCC-3′BHQ1
G2R_WA assay
(detection of Western African clade viruses)
G2R_WA forward primer
(SEQ ID NO: 4)
5′-CACACCGTCTCTTCCACAGA
G2R_WA reverse primer
(SEQ ID NO: 5)
5′-GATACAGGTTAATTTCCACATCG
G2R_WA probe
(SEQ ID NO: 11)
5′HEX-AACCCGTCGTAACCAGCAATACATTT-3′BHQ1
Human RNase P (RP)
https://www.ncbi.nlm.nih.gov/pmc/articles/PMC8669853/
RP-F
Human RNAse P gene Forward primer
(SEQ ID NO: 7)
AGATTTGGACCTGCGAGCG
RP-R
Human RNAse P gene Reverse primer
(SEQ ID NO: 8)
GAGCGGCTGTCTCCACAAGT
RP-P Human RNAse P gene Probe
(SEQ ID NO: 9)
Cy5-TTCTGACCT/ZEN/GAAGGCTCTGCGCG-3IABKFQ

IPC-MPXV V1.0 ratio 1:1:1 G2R_G assay, G2_WA ass, C3L assay, and Spacers

(SEQ ID NO: 12)
ctaatGGAAAGTGTAAAGACAACGAATACAGAAGCCGTAATCTATGTTGT
CTATCGTGTCCTCCGGGAACTTACGCTTTCAGATTATGTGATAGCtcatg
GATACAGGTTAATTTCCACATCGATATAGTTAAATGTATTGCTGGTTACG
ACGGGTTCGCATTTATCTGTGGAAGAGACGGTGTGttgttGGCATCTCCG
TTTAATACATTGATTAAAGAGTGTCCATCCGGTACCGGTACATTTAGCAT
ATATGGGTCCCATTTTTTGCTTTCTGTATCCAGGTAGACAcatgt

length: 295 bp

### DNA Amplification and Detection

Description* Update or generate new SOP RND-SOP-MPXV0001 for CLIA QMS*. Is extraction control available? Is RNAseP positive in negative and positive controls?

Each plate includes a positive MPXV control, a negative control, and detection of the internal housekeeping gene RNAseP for each sample

Final Concentrations of Primers are shown below in Table 24.

TABLE 24

| | Working | Final conc. |
|---|---|---|
| Primers and Probes | conc. stock | in reaction |
| G2R_G Forward primer | 10 uM | 400 nM |
| G2R_G Reverse primer | 10 uM | 400 nM |
| G2R_WA Forward primer | 10 uM | 400 nM |
| G2R_WA Reverse primer | 10 uM | 400 nM |
| C3L Forward primer | 10 uM | 400 nM |
| C3L Reverse primer | 10 uM | 400 nM |
| G2R_G-FAM probe | 10 uM | 200 nM |
| G2R_WA-FAM probe | 10 uM | 200 nM |
| CL3 FAM probe | 10 uM | 200 nM |

Preparation of Master Mix for Renegade duplex (N1 and RP) RT-PCR assay is shown in Table 25. Thermocycling conditions are summarized in Table 26. Table 27 summarizes the workflow interpretation.

TABLE 25

| Components | 1 × reaction | N × reaction |
|---|---|---|
| G2R_G Primers | 0.8 uL Each (1.6 uL total) | N × 0.8 uL |
| G2G_WA Primers | 0.8 uL Each (1.6 uL total) | N × 0.8 uL |
| RNAse P Primers | 0.8 uL Each (1.6 uL total) | N × 0.8 uL |
| G2R_G Probe-FAM | 0.4 uL | N × 0.4 uL |
| G2R_WA Probe-HEX | 0.4 uL | N × 0.4 uL |
| RNAse P Probe-Cy5 | 0.4 uL | N × 0.4 uL |
| TaqPath 1-Step Master Mix | 5 uL | N × 5 uL |
| H₂O | 4 uL | N × 4 uL |
| Total volume | 15 uL | N × 15 uL |

TABLE 26

| Step | # of cycles | Temperature | Time |
|---|---|---|---|
| Incubation | 1 | 25° C. | 2 min |
| Reverse transcription | 1 | 50° C. | 15 min |
| RT inactivation/initial | 1 | 95° C. | 2 min |
| denaturation | | | |
| Amplification | 40 | 95° C. | 3 sec |
| | | 60° C. | 30 sec |

TABLE 27

| Ct Values | | | |
|---|---|---|---|
| G2R_G | | Human RNase | |
| (Ct ≤ | G2R_WA | P (RP) | |
| 38) | (Ct ≤ 38) | (Ct ≤ 38) | Result Interpretation |
| <=38 | >38 or neg | ≤38 | MPXV Detected |
| <=38 | <=38 | any | MPXV, West African Clade Detected |
| neg | neg | ≤38 | MPXV Not Detected |
| <=38 | >38 or neg | >38 or | repeat, consult with |
| | | neg | supervisor/director |
| neg | neg | >38 | repeat, if same, result as Inconclusive |
| >38 or | any | any | repeat, consult with |
| neg | | | supervisor/director, |
| | | | if still >38, result as |
| | | | Equivocal |

TABLE 28
SUMMARY

| Performance | | | Acceptance |
|---|---|---|---|
| characteristics | Observed | Expected | criteria met? |
| Accuracy | (15/15) 100% | ≥95% | Yes |
| Precision | (89/90) 99% | ≥95% | Yes |
| Sensitivity (LoD) | (19/20) 95% at 74.2 cp/mL | ≥95% | Yes |
| Specificity | 100% (in silico analysis) | ≥95% | Yes |

| | |
|---|---|
| Reportable Range | Detected and Not Detected |
| Reference Range | Not Detected |
| Sample stability | Lesion swab in Zymo DNA/RNA |
| | Shield at 4° C. stable for 7 days |
| Contamination | No contamination was detected |

### Example 2: Introduction of DNAse Treatment to Determine Presence of mRNA Versus DNA Only

This assay was developed to prevent potential False Negative results. The protocol is designed to implement the molecular detection of MPXV in clinical samples. There are three targets. Two targets are virus-specific and probes are allocated within DNA-binding phosphoprotein (2) (13L) and Protein F11 (F11L) genes of MXPV. The assay configuration does not allow clade-specific detection but is designed to be mutation/deletion resistant due to independently targeting two MXPV conservative areas of the genome. The third probe is used as an indigenous control and targets human RNase P gene area.

### Material, Equipment, and Safety

PPE: disposable anti fluid gown, latex gloves, goggles or full-face cover, lab hat, and shoe covers, and for the elimination of used PPE.

Materials for decontamination of surfaces:

70% Ethanol/Isopropyl Alcohol

DI Water

Rnase Away

10% Bleach solution

Reagents:

DEPC-treated Water/Nuclease-Free Water

TaqPath qRT-PCR master mix

Forward/Reverse primers; I3L, F11L, & RNase P specific primers

FAM, TAMRA, and Cy5 probe

Zymo Research Kit—Quick DNA/RNA Viral MagBead (REF: R2141)

80% Ethanol

Nuclease Free water

100% Isopropanol/2-Propanol

Consumables:

KingFisher DEEP-WELL (DW) 96 Plate (REF:95040450)

KingFisher 96 Tip Comb for DW Magnets (REF: 97002534)

KingFisher 96 plate 200 uL (REF: 97002540)

Coming Incorporated 100 mL Reagent Reservoir (REF: 4872)

Biotix 25 mL DisposableReagent Reservoir (REF: SD-0025-5SWM)

300/1250 uL Integra pipette tips

MicroAmp Fast Optical 96-Well Reaction Plate with Barcode (0.1 mL) (REF: 4346906) 5.4.8. MicroAmp Optical Adhesive Film (REF: 4311971)

MicroPlate SealPlate Film

BIO-RAD Microseal ‘F’ Foil Seals (CAT: MSF1001)

Equipment:

QuantStudio 6 Flex PCR Machine

Kingfisher Extraction Machine

Temperature Control Centrifuge

300 uL Integra Mini 96

1250 uL Integra Voyager 8-channel

1250 uL Integra Voyager 6-channel

Supplies:

Transport containers

Specimen collection bags and triple packaging

Coolers and cold pack or dry ice

Labels and permanent markers

### Specimen Collection, Transport, and Storage

Acceptable Specimen Types are lesion swabs, resuspended in 1 mL of DNA/RNA shield.

This protocol pertains to accessioned patient specimens that meet “Accession SOP” detailed in CLIN-SOP-001—Sample Receipt and Sample Accession.

### Specimen Collection

Recommended specimen collection for monkeypox is skin lesion swabs of lesion surface and/or exudate, roofs from more than one lesion, or lesion crusts. Alternatively, swabs placed in viral transport media (VTM), specifically DNA/RNA shield for chemical inactivation, can be used.

Specimens should be stored refrigerated or frozen within an hour of collection and transported to the laboratory as soon as possible after collection. If transport exceeds seven days for the sample to be tested, specimens should be stored at −70° C. or lower.

Specimen Storage

Specimens can be stored at 2 to 8° C. for up to 72 hours after collection. If a delay in extraction is expected, store specimens at −70° C. or lower

Frozen samples should not be thawed more than once; repeated freeze-thaw cycles can lead to degradation of RNA and reduced viral titers.

After processing, purified RNA from patient test specimens is used in a subsequent RT-PCR assay, or stored −20° C. or lower. They may be stored short-term at 4° C.

### Procedure

### Reagent and Control Preparation

### Primer Probe Preparation

QC spec for primers: Standard desulting is sufficient but HPLC-purification is preferred if ordered in bulk (more than 25 nM). QC spec for probes: If ordered in IDT then all probes will be confirmed by mass-spec and HPLC purified by default. Upon arrival all primers and probes need to be resuspended to 100 uM concentration in TE pH=8.0 buffer.

TABLE 29
Primer/Probe Sequences

| | |
|---|---|
| F11L forward primer | 5′-CTTGACAGATATTGGACCGAACT-3′ (SEQ ID NO: 13) |
| F11L reverse primer | 5′-TGGGCATAGTGCAGGATTTAC-3′ (SEQ ID NO: 14) |
| F11L Probe | 5′TAMN-ACCTGGAATGTAAAGCCCTGAAACCC-3′AbRQSp |
| | (SEQ ID NO: 15) |
| I3L forward primer | 5′-AGTTTCCGCCGAAAGACTATG-3′ (SEQ ID NO: 16) |
| I3L reverse primer | 5′-GCGGCTAGTCCTATGTTGTATC-3′ (SEQ ID NO: 17) |
| I3L probe | 5′FAM-TGCGTTCCT/ZEN/TGTGTGTAATGTTTCCA-3′IABKFQ |
| | (SEQ ID NO: 18) |
| RP-F Human RNase P | 5′-AGATTTGGACCTGCGAGCG-3′ (SEQ ID NO: 7) |
| gene Forward primer | |
| RP-R Human RNase P | 5′-GAGCGGCTGTCTCCACAAGT-3′ (SEQ ID NO: 8) |
| gene Reverse primer | |
| RP-P Human RNase P | 5′Cy5-TTCTGACCT/ZEN/GAAGGCTCTGCGCG-3′IABKFQ |
| gene Probe | (SEQ ID NO: 9) |

Prepare aliquots of all primer and probes and store them at −20 C freezer. The working concentration stock is 100 uM for F11L forward and reverse primer, I3L forward and reverse primer, RNase P forward and reverse primers, F11L probe, I3L probe, and RNase P probe. All Primers and probes can vary in concentration, so to achieve 100 uM.

### Positive Target Control (PTC) Preparation (High/Low)

For each run a positive target control must be included. The positive target control is gBlock MPXV-IPC-V4 including: E3L, F11L, I3L, WA, G, G2, G3, C3L (MPXV Internal positive control).

When gBlock is ordered no special modifications are needed. Upon arrival resuspend it to 10 ng/ul concentration in TE pH=8.0 buffer. Then dilute it to MKPX-IPC-V4.0-1:10\hat{ }-7 by volume and use it as the high positive control (Ct˜28). For the low positive it can then be diluted to 1:10\hat{ }-8 (Ct˜32). This control is needed to verify PCR reagent integrity as well as proper assay set-up of the RT

PCR reactions for the F11L, I3L, and RNase P genes.

Positive Extraction Control (PEC) Preparation

For each run a positive extraction control must be included. The positive control is made from ATCC product “Quantitative Synthetic Monkeypox virus DNA” (P #: VR-3270SD). It arrives with a volume of 100 uL with a specification range of ≥1×105 to 1×106 copies/μL. This is then diluted to a working stock concentration of ˜670 copies/uL using Ix concentration DNA/RNA Shield. This control monitors quality of isolated RNA and malfunction in reagents.

The synthetic DNA used for the PEC has a variable concentration; target ct range for PEC should be around 30-35. Some adjustments may be necessary to reach this range.

Negative Extraction Control (NEC) Preparation

For each run, no template control must be included. It is needed to check for contamination of RT-PCR assay reagents and extraction.

Use sterile, nuclease-free water.

Assay

Lesion swabs from patients with suspected Monkeypox virus are resuspended in 1 mL of 1× DNA shield, and 300 uL are used in Kingfisher extractions.

Perform Kingfisher DNA/RNA extraction on both patient samples and extraction controls. Extraction controls should be added following MPXV Plate map.

PEC: ‘Quantitative Synthetic Monkeypox virus DNA’ (P #: VR-3270SD) dilution.

NEC: Nuclease free water

PEC and NEC should be treated as patient samples and extracted in each batch processed through the Kingfisher equipment. Prepare all Wash, Ethanol, and Elution plates as stated in the above referenced SOP.

Place 55 uL of DNA/RNAse free Water in the Elution plate.

DNA/RNA extractions should be tested promptly after the extraction process is completed. May be stored short-term in 4° C. Long-term storage should be done at −20° C. or lower to minimize DNA/RNA degradation.

Establish Molecular Assays on total RNA and DNA samples.

Thaw qRT-PCR TaqPath reagents (Cat #A15300 or A15299). Thaw primer/probe mixes.

Set up qRT-PCR reaction following table 30.

TABLE 30
“Master Mix” Recipe

| | | Volume | | Final |
|---|---|---|---|---|
| | Con- | per | n = 500 | Con- |
| Reagent | centration | Reaction | reactions | centration |
| TaqPath Master Mix | 4x | 5 uL | 2.5 mL | 1x |
| F11L Forward Primer | 100 uM | 0.08 uL | 40 uL | 400 nM |
| FL Reverse Primer | 100 uM | 0.08 uL | 40 uL | 400 nM |
| I3L Forward Primer | 100 uM | 0.08 uL | 40 uL | 400 nM |
| I3L Reverse | 100 uM | 0.08 uL | 40 uL | 400 nM |
| RNase P Forward Primer | 100 uM | 0.08 uL | 40 uL | 400 nM |
| RNase P Reverse Primer | 100 uM | 0.08 uL | 40 uL | 400 nM |
| I3L-FAM Probe | 100 uM | 0.04 uL | 20 uL | 200 nM |
| F11L-TAMRA Probe | 100 uM | 0.04 uL | 20 uL | 200 nM |
| RNase P-Cy5 Probe | 100 uM | 0.04 uL | 20 uL | 200 nM |
| DEPC-treated | | 9.4 uL | 4.7 mL | |
| DNAse/RNAse free | | | | |
| Water | | | | |
| | | 15 uL | 7.5 mL | 1x |

Following addition of 15 uL of 1× Master Mix to each well, add 5 uL of each purified DNA/RNA sample. The total reaction volume is 20 uL.

Cover plate and add to Machine, briefly centrifulge, aligning A1 well to A1 plate marker.

Thermocycler Protocol for the PCR run method is shown in Table 31.

TABLE 31

| Temperature | Hold time | Number of Cycles |
|---|---|---|
| 25° C. | 2 minutes | x1 |
| 50° C. | 15 minutes | x1 |
| 95° C. | 2 minutes | x1 |
| 95° C. | 3 seconds | X40 * Fluorescence |
| 60° C. | 30 seconds | Acquired at this step |

Baseline is set to “AUTO”

Thresholds are set to 0.05 for each target.

Run assay and interpret results.

Interpretation of Results for Quality Control and Actions: The controls for the RT-PCR for detecting Monkeypox (F11L & I3L) are evaluated using nucleic acid amplification curve and Ct values generated by the RT-PCR system software. See table 32 for valid control results. If controls do not meet requirements, then they must be reprocessed. Table 33 shows the interpretation of results.

TABLE 32

| ≥≤ | F11L | I3L | RNase P | Result Interpretation |
|---|---|---|---|---|
| PEC | ≤37.0 | ≤37.0 | Any Value | Passed |
| NEC | >37.0 | >37.0 | >37.0 | Passed |
| PTC | ≤37.0 | ≤37.0 | Any Value | Passed |

Ct values in the FAM, TAMRA, and CY5 channels for a valid Negative Extraction Control (NEC) should be “Undetermined” or ≥37.0 and there should be no sigmoidal amplification.

TABLE 33

| F11L | I3L | RNase P | Result Interpretation |
|---|---|---|---|
| ≤37 | ≤37 | Any value | MPXV Detected |
| >37 or | >37 or | ≤37 | MPXV Not Detected |
| UND | UND | | |
| neg | neg | >37 | Repeat, if same, result as Inconclusive |
| >37 or | ≤37.0 | Any value | Repeat, Consult with Supervisor/Director, |
| UND | | | if same, result as Equivocal |
| ≤37.0 | >37 or | Any value | Repeat, Consult with Supervisor/Director, |
| | UND | | if same, result as Equivocal |

### Result Interpretation

As Ct cutoff is set at 37.0, all valid amplification must occur within the 0.05 threshold criteria to be considered valid. Results are to be used in conjunction with patient medical history by requesting provider. Results alone are not to be used as the sole indication of infection. According to the International Health Regulations (IHR), all monkeypox confirmed cases should be notified within 24 hours through official IHR channels.

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the specific embodiments of the invention described herein may be employed in practicing the invention. It is intended that the following claims define the scope of the invention and that methods and structures within the scope of these claims and their equivalents are covered thereby.

1. An assay for detecting a virus in a biological sample, wherein the assay comprises a reverse transcription PCR assay that coverts viral messenger RNA to produce DNA; and amplifies the produced DNA in the presence of viral DNA.

2. The assay of claim 1 wherein the virus is an orthopoxvirus.

3. The assay of claim 1 wherein the virus is the causative agent of Monkeypox.

4. The assay of claim 1 wherein a generic orthopox virus primer set and additional primers for clade-specific regions are included in the assay.

5. The assay of claim 4 wherein the additional primers allow for the determination of West African monkeypox virus or Congo Basin monkeypox virus.

6. The assay of claim 1 wherein two targets are virus-specific targeting two monkeypox conservative areas of the genome probes and an indigenous control targeting human RNase P gene area.

7. The assay of claim 6 wherein the virus-specific targeting probes are allocated within DNA-binding phosphoprotein (2) (I3L) and Protein F11 (F11L) genes of the monkeypox virus.

8. A method for detecting a virus in a biological sample, wherein the method comprises converting viral messenger RNA to produce DNA in a reverse transcription PCR assay; and
amplifying the produced DNA in the presence of viral DNA.

9. The method of claim 8 wherein the virus is an orthopoxvirus.

10. The method of claim 8 wherein the virus is the causative agent of Monkeypox.

11. The method of claim 8 wherein a generic orthopox virus primer set and additional primers for clade-specific regions are included in the assay.

12. The method of claim 11 wherein the additional primers allow for the determination of West African monkeypox virus or Congo Basin monkeypox virus.

13. The method of claim 8 wherein two targets are virus-specific targeting two monkeypox conservative areas of the genome probes and an indigenous control targeting human RNase P gene area.

14. The method of claim 13 wherein the virus-specific targeting probes are allocated within DNA-binding phosphoprotein (2) (I3L) and Protein F11 (F11L) genes of the monkeypox virus.


SEQUENCE LISTING (18 sequences)

SEQ ID NO: 1 - DNA, 26 bp, synthetic construct
  source 1..26: mol_type = other DNA
ggaaaatgtaaagacaacgaatacag

SEQ ID NO: 2 - DNA, 24 bp, synthetic construct
  source 1..24: mol_type = other DNA
gctatcacataatctggaagcgta

SEQ ID NO: 3 - DNA, 21 bp, synthetic construct
  source 1..21: mol_type = other DNA
  modified_base 1: mod_base = OTHER; note = 5' Fluorescein-aagccgtaa Zen quencher modified nucleotide
  modified_base 21: mod_base = OTHER; note = 3' Iowa Black FQ modified nucleotide
tctatgttgtctatcgtgtcc

SEQ ID NO: 4 - DNA, 20 bp, synthetic construct
  source 1..20: mol_type = other DNA
cacaccgtctcttccacaga

SEQ ID NO: 5 - DNA, 23 bp, synthetic construct
  source 1..23: mol_type = other DNA
gatacaggttaatttccacatcg

SEQ ID NO: 6 - DNA, 26 bp, synthetic construct
  source 1..26: mol_type = other DNA
  modified_base 1: mod_base = OTHER; note = 5' Hexachlorofluorescein modified nucleotide
  modified_base 26: mod_base = OTHER; note = 3' Iowa Black FQ modified nucleotide
aacccgtcgtaaccagcaatacattt

SEQ ID NO: 7 - DNA, 19 bp, synthetic construct
  source 1..19: mol_type = other DNA
agatttggacctgcgagcg

SEQ ID NO: 8 - DNA, 20 bp, synthetic construct
  source 1..20: mol_type = other DNA
gagcggctgtctccacaagt

SEQ ID NO: 9 - DNA, 14 bp, synthetic construct
  source 1..14: mol_type = other DNA
  modified_base 1: mod_base = OTHER; note = 5' Cyanine 5 dye-ttctgacct Zen quencher modified nucleotide
  modified_base 14: mod_base = OTHER; note = 3' Iowa Black FQ modified nucleotide
gaaggctctgcgcg

SEQ ID NO: 10 - DNA, 30 bp, synthetic construct
  source 1..30: mol_type = other DNA
  modified_base 1: mod_base = OTHER; note = 5' Fluorescein modified nucleotide
  modified_base 30: mod_base = OTHER; note = 3' Black hole quencher 1 modified nucleotide
aagccgtaatctatgttgtctatcgtgtcc

SEQ ID NO: 11 - DNA, 26 bp, synthetic construct
  source 1..26: mol_type = other DNA
  modified_base 1: mod_base = OTHER; note = 5' Hexachlorofluorescein modified nucleotide
  modified_base 26: mod_base = OTHER; note = 3' Black hole quencher 1 modified nucleotide
aacccgtcgtaaccagcaatacattt

SEQ ID NO: 12 - DNA, 295 bp, synthetic construct
  source 1..295: mol_type = other DNA
ctaatggaaagtgtaaagacaacgaatacagaagccgtaatctatgttgtctatcgtgtcctccgggaacttacgctttcagattatgtgatagctcatggatacaggttaatttccacatcgatatagttaaatgtattgctggttacgacgggttcgcatttatctgtggaagagacggtgtgttgttggcatctccgtttaatacattgattaaagagtgtccatccggtaccggtacatttagcatatatgggtcccattttttgctttctgtatccaggtagacacatgt

SEQ ID NO: 13 - DNA, 23 bp, synthetic construct
  source 1..23: mol_type = other DNA
cttgacagatattggaccgaact

SEQ ID NO: 14 - DNA, 21 bp, synthetic construct
  source 1..21: mol_type = other DNA
tgggcatagtgcaggatttac

SEQ ID NO: 15 - DNA, 26 bp, synthetic construct
  source 1..26: mol_type = other DNA
  modified_base 1: mod_base = OTHER; note = 5' carboxytetramethylrhodamine N-hydroxysuccinimide ester modified nucleotide
  modified_base 26: mod_base = OTHER; note = 3' Iowa black RQ quencher modified nucleotide
acctggaatgtaaagccctgaaaccc

SEQ ID NO: 16 - DNA, 21 bp, synthetic construct
  source 1..21: mol_type = other DNA
agtttccgccgaaagactatg

SEQ ID NO: 17 - DNA, 22 bp, synthetic construct
  source 1..22: mol_type = other DNA
gcggctagtcctatgttgtatc

SEQ ID NO: 18 - DNA, 17 bp, synthetic construct
  source 1..17: mol_type = other DNA
  modified_base 1: mod_base = OTHER; note = 5' Fluorescein-tgcgttcct Zen quencher modified nucleotide
  modified_base 17: mod_base = OTHER; note = 3' Iowa Black FQ modified nucleotide
tgtgtgtaatgtttcca